SEQUENCE LISTING (4 sequences)

SEQ ID NO: 1 - RNA, 1467 nt, synthetic construct
  source 1..1467: mol_type = other RNA
ttgtggaaggaaagggctttattcagctgggagcaccggcggactcacgtctccaaaaaccgagctccccgagcgagcaattcctgtcccttttaagggcttacagctctaagggggtccgtgtgagagggtcgtgatcgattgagcaaccagcgggtacgtgactgcgggctgcacgcaccggtaatcagaacagagcagaacaggccagggattttcacgatgcttttccatacaatgtctggaatctatttgggaagctgaggcaggagaattgcttgaatccaggaggcggaggttgcagtgagccaagatcataccattgcactccagcctgggcaacaaaagcgaaactctctctgggcctcaggctcctcatctataaaatggggacatgcaagtccctaacccagaaggtcagtgtggggatcgaacaggagatagcacatgaagagcacaggttgagtttgtggggtgcgggggcgtttggtaggacagtcagtggtttaccaaaccaaagtgcaagctgaaagtttcggaccccagggatcagtgaaagagcttaaagagcccagtctcagggatcagtgcccacaaaggtctggaattctggatgtcaggataaagagagttgacagggagactgagattcaccctcaccctacacggggatgttttcctctgaccccttttagcttccttttttggactgcacgtggtctcagaacggggcctccacagcccaggcgaggtggtctgtggacatggctcctgtaagtgcccaccagacagagtgtgaggcagccaagctgaggtcggcactggaagagcccaagagaaagcttgggaggtccatggtggccactgaagtccagaagacaagacagacttaggcaggccagaagggaaactactcctctgtgaggggctgggaggacagaggaggagcccaggtgtcctgtggagaactgaatttcagaagaatttcttgggggctctggagcgccccgcagaaccacctcaaggcaagagcaggaaccaagaggcctctgagttcttgctgtatgacttggaaaaatgttccgcccctccctgggcctcgtttcctccactgtatgatgagggagtgaactctgagatcacgtctatatcgatcaccatcattccatgtgacctagcacagacgatgttggttcctgcaggcacaaggatatggctgcttctcttgggctgggctgtctcacaagcaggctcagtcacaaggaatgaagtagtctatctgcaaggtggtcccaagtgacatcagcagaggatcttgacatcctgtgggcgcctgcagatgctggctctttctctcctcccagggataagaccgcacttctctcactggtcccagtgggcatagcaaaagcaacaaagctgtggggcttgttggtgcttcatcgaacaggcctaggcaggaggccc

SEQ ID NO: 2 - DNA, 1467 bp, synthetic construct
  source 1..1467: mol_type = other DNA
gggcctcctgcctaggcctgttcgatgaagcaccaacaagccccacagctttgttgcttttgctatgcccactgggaccagtgagagaagtgcggtcttatccctgggaggagagaaagagccagcatctgcaggcgcccacaggatgtcaagatcctctgctgatgtcacttgggaccaccttgcagatagactacttcattccttgtgactgagcctgcttgtgagacagcccagcccaagagaagcagccatatccttgtgcctgcaggaaccaacatcgtctgtgctaggtcacatggaatgatggtgatcgatatagacgtgatctcagagttcactccctcatcatacagtggaggaaacgaggcccagggaggggcggaacatttttccaagtcatacagcaagaactcagaggcctcttggttcctgctcttgccttgaggtggttctgcggggcgctccagagcccccaagaaattcttctgaaattcagttctccacaggacacctgggctcctcctctgtcctcccagcccctcacagaggagtagtttcccttctggcctgcctaagtctgtcttgtcttctggacttcagtggccaccatggacctcccaagctttctcttgggctcttccagtgccgacctcagcttggctgcctcacactctgtctggtgggcacttacaggagccatgtccacagaccacctcgcctgggctgtggaggccccgttctgagaccacgtgcagtccaaaaaaggaagctaaaaggggtcagaggaaaacatccccgtgtagggtgagggtgaatctcagtctccctgtcaactctctttatcctgacatccagaattccagacctttgtgggcactgatccctgagactgggctctttaagctctttcactgatccctggggtccgaaactttcagcttgcactttggtttggtaaaccactgactgtcctaccaaacgcccccgcaccccacaaactcaacctgtgctcttcatgtgctatctcctgttcgatccccacactgaccttctgggttagggacttgcatgtccccattttatagatgaggagcctgaggcccagagagagtttcgcttttgttgcccaggctggagtgcaatggtatgatcttggctcactgcaacctccgcctcctggattcaagcaattctcctgcctcagcttcccaaatagattccagacattgtatggaaaagcatcgtgaaaatccctggcctgttctgctctgttctgattaccggtgcgtgcagcccgcagtcacgtacccgctggttgctcaatcgatcacgaccctctcacacggacccccttagagctgtaagcccttaaaagggacaggaattgctcgctcggggagctcggtttttggagacgtgagtccgccggtgctcccagctgaataaagccctttccttccacaa

SEQ ID NO: 3 - DNA, 23 bp, synthetic construct
  source 1..23: mol_type = other DNA
agatcctctgctgatgtcacttg

SEQ ID NO: 4 - DNA, 18 bp, synthetic construct
  source 1..18: mol_type = other DNA
aaaatgttccgcccctcc

(19) United States
(12) Patent Application Publication
(10) Pub. No.: US 2024/0263178 A1
(43) Pub. Date: Aug. 8, 2024

(54) Use Of LncRNA DCRT As Drug And Its Drug For Treating Cardiovascular Diseases, Screening Method, And Preparation Method Thereof

(71) Applicant: Tongji Hospital, Tongji Medical College, Huazhong University of Science and Technology, Wuhan City (CN)

(72) Inventors: Chen CHEN, Wuhan City (CN); Huaping LI, Wuhan City (CN); Daowen Wang, Wuhan City (CN); Yanru Zhao, Wuhan City (CN); Hengzhi DU, Wuhan City (CN)

(21) Appl. No.: 18/419,196

(22) Filed: Jan. 22, 2024

(30) Foreign Application Priority Data
Feb. 6, 2023 (CN) 202310099726.4

(51) Int. Cl. C12N 15/113; A61K 31/7105; A61P 9/00; C12N 15/86; C12Q 1/6883

(52) U.S. Cl. CPC C12N 15/113; A61K 31/7105; A61P 9/00; C12N 15/86; C12Q 1/6883; C12N 2750/14143; C12Q 2600/136

(57) ABSTRACT

The present invention of use of lncRNA DCRT as drug and its drug for treating cardiovascular diseases, screening method, and preparation method thereof belongs to the field of biomedicine. The present invention provides use of lncRNA DCRT with sequence shown as SEQ ID NO. 1 as drug. The present invention further provides a new drug for treating cardiovascular diseases with lncRNA DCRT as target. Based on lncRNA DCRT, the present invention also provides a screening method and preparation method of drug for treating dilated cardiomyopathy. The present invention confirms through animal experiments that upregulation of lncRNA DCRT has effect of improving central function impairment and pathological increase in myocardial cell area in a cardiac dilation model induced by thoracic aortic constriction surgery.

### CROSS-REFERENCE TO RELATED APPLICATION

This application claims priority to and the benefit of Chinese patent Application No. 202310099726.4 filed Feb. 6, 2023. The entire contents of Chinese patent Application No. 202310099726.4 and the English translation of Chinese patent Application No. 202310099726.4 are incorporated herein by reference.

### SEQUENCE LISTING

This application contains references to amino acid sequences and/or nucleic acid sequences which have been submitted concurrently herewith as the sequence listing .xml file entitled “000005 us_SequenceListing.xml”, file size 7,712 bytes, created on Jan. 21, 2024. The aforementioned sequence listing is hereby incorporated by reference in its entirety pursuant to 37 C.F.R. § 1.52(e)(5).

### TECHNICAL FIELD

The present invention belongs to the pharmaceutical field of cardiovascular diseases, and specifically relates to use of LncRNA DCRT as drug and its drug for treating cardiovascular diseases, screening method, and preparation method thereof.

### BACKGROUND

Heart failure is a complex syndrome caused by impaired ventricular filling and/or ejection capacity led by various structural or functional diseases of heart, which significantly increases risk of hospitalization and death worldwide. Despite advances in scientific research and treatment strategies, treating heart failure remains a challenge. Dilated cardiomyopathy is one of the most common causes of heart failure, it's defined as left ventricular systolic dysfunction and dilation under abnormal load conditions proportional to degree of left ventricular damage or without coronary artery disease. Heart failure is a clinical syndrome that progresses to the terminal stage of different cardiovascular diseases, with poor prognosis and high mortality rate, which is one of the main causes threatening human health and increasing medical burden. Despite progress in recent treatment, a large number of patients with dilated cardiomyopathy still experience arrhythmias, heart failure, and sudden cardiac death. Dilated cardiomyopathy ranks third among various cardiovascular diseases that lead to heart failure and is the leading cause of heart transplant surgery.

For pathogenesis of dilated cardiomyopathy, genetic mutations involving genes account for 35% of cases. Endocrine abnormalities, drugs and toxins, as well as infectious factors, are all causes of acquired dilated cardiomyopathy. Importantly, mitochondrial dysfunction plays a crucial role in pathogenic process of dilated cardiomyopathy. Mitochondria are “power source” of eukaryotic cells, a highly dynamic organelle that is particularly important for cells that require high energy, such as cardiomyocytes. Mitochondria provide a large amount of adenosine triphosphate every day, which maintains heart function through oxidative phosphorylation. Mutations in genes encoding mitochondrial proteins often lead to abnormal oxidative phosphorylation, which not only results in ATP deficiency but also increases ROS production and changes in antioxidant defense system, ultimately promoting development of dilated cardiomyopathy. Therefore, maintaining normal mitochondrial function in myocardial cells can serve as a new target for treating heart failure.

Long chain non coding RNAs are a newly discovered class of non coding RNAs with nucleotide sequences greater than 200 nt, which play an important role in regulating gene expression. Recently, an increasing number of studies have found that lncRNA plays an important role in various cardiovascular diseases, including myocardial metabolism, myocardial hypertrophy, cardiac conduction system, and cardiac development. Clarifying molecular mechanisms underlying function of lncRNA is a major challenge in this field. In recent years, more and more studies have shown that lncRNA is involved in various processes of gene expression in disease process, including transcriptional regulation, post transcriptional regulation, epigenetic regulation and etc. The function of lncRNA can be regulated by cis- or trans-action to regulate expression level of downstream target genes. Mechanism of lncRNAs located in different subcellular structures is different. Localization in cytoplasm and nucleus of lncRNA helps us determine the action mode of lncRNA. The lncRNA located in the nucleus may affect gene transcription through various mechanisms, including epigenetic modifications, interactions with transcription factors, and affecting mRNA processing or output. Action modes of lncRNAs distributed in the cytoplasm mainly include: affecting stability of mRNA, affecting translation initiation, acting as competitive endogenous RNAs, or affecting post translation modifications, etc. Due to lncRNAs lack conservation among genuses, although many studies have reported the function and mechanism of long-chain non coding RNAs in mouse models, little is known about their roles in human heart. There is currently no report of an association between lncRNA DCRT and cardiovascular disease.

### SUMMARY

Based on the objective problems and shortcomings in this field, the present invention accidentally discovered relationship between lncRNA DCRT and mechanism of dilated cardiomyopathy, and further conducted molecular experiments to develop an anti-dilated cardiomyopathy drug with lncRNA DCRT as the drug target. At the same time, animal experiments were conducted to verify its effectiveness, and it was found that molecules targeting lncRNA DCRT and upregulating expression of DCRT can significantly improve cardiac function impairment and pathological increase in myocardial cell area in dilated cardiomyopathy.

Technical solution of the present invention is as follows:
Use of lncRNA DCRT with sequence shown as SEQ ID NO.1 as drug.
A reverse complementary sequence of lncRNA DCRT is shown as SEQ ID NO.2; preferably, the drug refers to a drug for treating cardiovascular disease.

The cardiovascular disease is dilated cardiomyopathy.

A drug for treating cardiovascular disease, characterized in that, its active pharmaceutical ingredient is taking lncRNA DCRT with a sequence shown as SEQ ID NO.1 as target.
the active pharmaceutical ingredient comprises: a substance regulating expression of lncRNA DCRT.
Said regulating refers to up-regulating active pharmaceutical ingredient active pharmaceutical ingredient;

Preferably, the substance regulating expression of lncRNA DCRT is selected from a group consisting of primers upregulating or overexpressing lncRNA DCRT, a recombinant expression vector upregulating or overexpressing lncRNA DCRT, and a transformant upregulating or overexpressing lncRNA DCRT;
preferably, primers upregulating or overexpressing lncRNA DCRT include:

forward primer:
(SEQ ID NO: 3)
5′-AGATCCTCTGCTGATGTCACTTG-3′,
reverse primer:
(SEQ ID NO: 4)
5′-AAAATGTTCCGCCCCTCC-3′;

preferably, the recombinant expression vector upregulating or overexpressing lncRNA DCRT refers to an expression vector that connects amplification products of primers upregulating or overexpressing lncRNA DCRT; preferably, the expression vector is selected from: pAAV vector;
preferably, the transformant upregulating or overexpressing lncRNA DCRT is host transformed with the recombinant expression vector;
preferably, the host is selected from recombinant adenovirus or cell;
preferably, the drug further comprises: medicinal excipient;
preferably, the cardiovascular disease is dilated cardiomyopathy.

A method of preparing a drug for treating cardiovascular disease, characterized in that, comprising the following step: a substance upregulating or overexpressing lncRNA DCRT is used as the active pharmaceutical ingredient of the drug; sequence of lncRNA DCRT is shown as SEQ ID NO.1 active pharmaceutical ingredient.

The method of preparing a drug for treating cardiovascular disease also comprising the following step: a substance upregulating or overexpressing lncRNA DCRT is mixed with a medicinal excipient to produce the drug;
preferably, the substance upregulating or overexpressing lncRNA DCRT is selected from a group consisting of primers upregulating or overexpressing lncRNA DCRT, a recombinant expression vector upregulating or overexpressing lncRNA DCRT, and a transformant upregulating or overexpressing lncRNA DCRT;
preferably, primers upregulating or overexpressing lncRNA DCRT include:

forward primer:
(SEQ ID NO: 3)
5′-AGATCCTCTGCTGATGTCACTTG-3′,
reverse primer:
(SEQ ID NO: 4)
5′-AAAATGTTCCGCCCCTCC-3′;

preferably, a recombinant expression vector upregulating or overexpressing lncRNA DCRT refers to an expression vector that connects amplification products of primers upregulating or overexpressing lncRNA DCRT;
preferably, the expression vector is selected from: pAAV vector;
preferably, the transformant upregulating or overexpressing lncRNA DCRT is host transformed with the recombinant expression vector;
preferably, the host is selected from recombinant adenovirus or cell;
preferably, the cardiovascular disease is dilated cardiomyopathy.

A screening method of a drug for treating cardiovascular disease, characterized in that, comprising the following step: screening candidate substance with lncRNA DCRT as target; sequence of lncRNA DCRT is shown as SEQ ID NO.1. The screening method of a drug for treating cardiovascular disease further comprising the following step: further screening substance regulating lncRNA DCRT expression from the candidate substance as active pharmaceutical ingredient of the drug active pharmaceutical ingredient;
preferably, said regulating refers to up-regulating;
preferably, the substance regulating lncRNA DCRT expression is selected from: substance upregulating or overexpressing lncRNA DCRT;
preferably, substance upregulating or overexpressing lncRNA DCRT is selected from: a group consisting of primers upregulating or overexpressing lncRNA DCRT, a recombinant expression vector upregulating or overexpressing lncRNA DCRT, and a transformant upregulating or overexpressing lncRNA DCRT;
preferably, primers upregulating or overexpressing lncRNA DCRT include:

forward primer:
(SEQ ID NO: 3)
5′-AGATCCTCTGCTGATGTCACTTG-3′,
reverse primer:
(SEQ ID NO: 4)
5′-AAAATGTTCCGCCCCTCC-3′;

preferably, a recombinant expression vector upregulating or overexpressing lncRNA DCRT refers to an expression vector that connects amplification products of primers upregulating or overexpressing lncRNA DCRT;
preferably, the expression vector is selected from: pAAV vector;
preferably, the transformant upregulating or overexpressing lncRNA DCRT is host transformed with the recombinant expression vector;
preferably, the host is selected from recombinant adenovirus or cell;
preferably, the cardiovascular disease is dilated cardiomyopathy.

The present invention provides a drug for treating dilated cardiomyopathy, wherein a active pharmaceutical ingredient of the drug targets lncRNA DCRT and exerts the therapeutic effect of treating dilated cardiomyopathy by upregulating expression of lncRNA DCRT.

Further configuration of the present invention is that the active pharmaceutical ingredient comprises a substance containing upregulated or overexpressed lncRNA DCRT, and sequence of the lncRNA DCRT is shown as SEQ ID NO.1.

By adopting above technical solution, the active pharmaceutical ingredient of the drug includes substance that upregulate or overexpress lncRNA DCRT, thereby increasing the expression level of lncRNA DCRT and further treating the disease.

Further configuration of the present invention is to further include pharmaceutically acceptable excipient.

By adopting above technical solution, a person skilled in the art can add various pharmaceutically acceptable excipient/excipient to the anti dilated cardiomyopathy drug of the present invention according to objective needs, and make various dosage forms for easy sales or promotion.

Further embodiments of the present invention include reagent for buffering, and/or synthesizing, and/or purifying the lncRNA DCRT.

The present invention also provides a drug for treating dilated cardiomyopathy, wherein the active pharmaceutical ingredient of the drug have degradation or inhibitory effect on substance that downregulate the expression of lncRNA DCRT sequence.

The present invention also provides a screening method for anti dilated cardiomyopathy drug, detecting whether candidate substance can upregulate the expression of lncRNA DCRT.

The present invention also provides a substance that can upregulate the expression of lncRNA DCRT for preparing anti dilated cardiomyopathy drug.

The present invention also provides a use of a full-length sequence fragment of lncRNA DCRT in preparing drug for treating dilated cardiomyopathy. Full-length sequence fragment of lncRNA DCRT is shown as SEQ ID NO.1.

Another aspect of the present invention provides a use of a substance upregulating the expression of lncRNA DCRT in preparing anti dilated cardiomyopathy drug. Any act of packaging above substance in commercial packaging labeled with anti dilated cardiomyopathy use for any scale falls within protection scope of the present invention.

The beneficial effects of the present invention are as follows:

The present invention innovatively discovered highly conserved lncRNA DCRT in the heart tissue of patients with dilated cardiomyopathy. lncRNA DCRT is mainly localized in nucleus of myocardial cells and significantly reduced in myocardial cells of patients with dilated cardiomyopathy. Subsequently, it's confirmed by animal experiments that upregulating lncRNA DCRT has an effect of improving central function impairment and pathological increase in myocardial cell area in a cardiac dilation model induced by thoracic aortic constriction surgery.

In order to achieve the therapeutic purpose of dilated cardiomyopathy, the present invention uses lncRNA DCRT in animal experiments. It has been confirmed in animal experiments that a synthetic of lncRNA DCRT can significantly improve cardiac function impairment and myocardial cell contraction dysfunction induced by thoracic aortic constriction, to achieve a goal of assisting in treating dilated cardiomyopathy. Therefore, based on above findings and results, the present invention provides a drug for clinically treating dilated cardiomyopathy with lncRNA DCRT as a therapeutic target.

Based on a base sequence of lncRNA DCRT, the present invention designs a sequence for expressing lncRNA DCRT, and synthesizes it. A use of lncRNA DCRT synthetic can significantly improve cardiac function impairment induced by thoracic aortic constriction, myocardial cell contractile dysfunction, and calcium ion instability. This indicates that lncRNA DCRT has a potential to improve dilated cardiomyopathy.

The present invention found through experiments that the expression of lncRNA DCRT in heart tissue of patients with dilated cardiomyopathy was reduced (FIG. 1A), and lncRNA DCRT was significantly reduced mainly in a myocardial cells of patients with dilated cardiomyopathy. It's found by in vivo experiments that increasing a full-length synthetic of lncRNA DCRT can significantly improve cardiac function impairment and pathological increase in myocardial cell area induced by thoracic aortic constriction, to achieve the goal of assisting in treating dilated cardiomyopathy.

### DESCRIPTION OF DRAWINGS

FIG. 1A shows a total expression level of lncRNA DCRT in heart tissues of patients with dilated cardiomyopathy and a control population in experiment example 1 of the present invention; it was found that content of lncRNA DCRT decreased in the patient samples; wherein, the vertical axis “Relative DCRT Level” refers to the relative expression level of lncRNA DCRT, the horizontal axis “Normal” refers to the control population, and DCM refers to the patients with dilated cardiomyopathy.

FIG. 1B shows an expression of lncRNA DCRT respectively in myocardial cells, fibroblasts, and endothelial cells in human heart tissue of experiment example 1 of the present invention in three row of photos from top to bottom. Among them, cTNT is a specific marker for myocardial cells, referring to myocardial cell staining; Col1a1 is a specific marker for fibroblasts, referring to fibroblast staining; CD31 is a specific marker for endothelial cells, referring to endothelial cell staining; DAPI refers to DAPI staining result graph of cell nucleus; Merge refers to a merging image of the first three images in the same row.

Expression detection methods of cTNT, Col1a1, and CD31, as well as DAPI staining operation, can refer to the methods and steps recorded in the article “LncRNA ZNF593-AS Alleviates Contract Dysfunction in Dilated Cardiopathy”.

FIG. 2 shows a therapeutic effect of lncRNA DCRT on dilated cardiomyopathy in experiment example 2 of the present invention; among them, the vertical axis of the left figure in FIG. 2A refers to ejection fraction, the vertical axis of the right figure refers to shortening fraction, GFP on the horizontal axis of the left and right figures refers to a negative control mice overexpressing only green fluorescent protein, and mDCRT refers to the mice overexpressing lncRNA DCRT; Sham refers to a sham surgery group mice, TAC refers to a aortic arch constriction (TAC) disease model mice;

Operation of overexpressing GFP, construction method of Sham mice, and construction method of TAC disease model mice can refer to the methods and steps recorded in the article “LncRNA ZNF593-AS Alleviates Contract Dysfunction in Dilated Cardiopathy”.

The vertical axis of the left figure in FIG. 2B refers to maximum rate of increase in left ventricular pressure (dp/dtmax), the vertical axis of the right figure refers to maximum rate of decrease in left ventricular diastolic pressure (dp/dtmin), GFP on the horizontal axis of the left and right figures refers to negative control mice overexpressing only green fluorescent protein, and mDCRT refers to mice overexpressing lncRNA DCRT; Sham refers to a sham surgery group mice, TAC refers to an aortic arch constriction (TAC) disease model mice;

FIG. 2C shows detection results of mouse myocardial cell area after high expression of lncRNA DCRT in mouse heart in experiment example 2 of the present invention; among them, the left figure is WGA staining result graph, and the right figure is bar graph of myocardial cell area; the mDCRT in the left figure refers to mice overexpressing lncRNA DCRT; Sham refers to sham surgery group mice, TAC refers to aortic arch constriction (TAC) disease model mice; the vertical axis in the right figure refers to myocardial cell area, the horizontal axis GFP refers to negative control mice overexpressing only green fluorescent protein, and mDCRT refers to mice overexpressing lncRNA DCRT.

### EMBODIMENTS

The technical solution of the present invention will be described clearly and completely in the following content by combining with specific examples and experimental examples. Obviously, the described experimental examples and examples are only a part of the present invention's experimental examples and examples, not all of them. Based on the experimental examples and examples of the present invention, all other examples obtained by a person skilled in the art without creative work fall within protection scope of the present invention.

### Instrument and Equipment

Nanodrop® ND-1000 nucleic acid analyzer
ABI 9700 PCR instrument
ABI 7900HT fluorescence real-time quantitative PCR instrument
Beckman X-15R Low Temperature High Speed Centrifuge

### Reagents and Consumables

TRIZOL was purchased from Invitrogen;

The reverse transcription kit was purchased from Thermofish Company;
SYBR Green was purchased from Thermofish Company;
The EasyPure Plasmid MiniPrep Kit plasmid extraction kit was purchased from Beijing TransGen Biotech Company.

### Sources of Biomaterials

Peripheral blood of patients with dilated cardiomyopathy comes from hospitalized patients at Tongji Hospital in Wuhan from 2012 to 2018, all of whom have signed informed consent forms;
C57BL/6 is from Jiangsu GemPharmatech biotechnology Company Limited.

Examples group 1. use of lncRNA DCRT as drug of the present invention

This group of examples provides use of lncRNA DCRT with sequence shown as SEQ ID NO.1 as drug.

In some embodiments, a reverse complementary sequence of lncRNA DCRT is shown as SEQ ID NO.2.

In specific embodiments, the drug refers to a drug for treating cardiovascular disease.

In more specific embodiments, the cardiovascular disease is dilated cardiomyopathy.

The present invention for the first time reveals the pharmaceutical use of lncRNA DCRT shown as SEQ ID NO.1. Based on disclosure of the present invention, those skilled in the art can receive guidance and inspiration to use lncRNA DCRT shown as SEQ ID NO.1 in pharmacy. Any act of synthesizing, amplifying, preparing, producing, using, expressing, secreting, enriching, propagating, selling, promising for sale, importing, or exporting lncRNA DCRT shown as SEQ ID NO.1 for medicinal or commercial purposes falls within protection scope of the present invention.

The drug is not limited to treating cardiovascular diseases. Based on the disclosed content of the present invention, a person skilled in the art can receive guidance and inspiration to use lncRNA DCRT shown as SEQ ID NO.1 as drug and treating diseases other than cardiovascular diseases. Any use of lncRNA DCRT shown as SEQ ID NO.1 as a drug target for treating any disease falls within protection scope of the present invention.

Examples group 2. drug for treating cardiovascular diseases of the present invention.

This group of examples provides a drug for treating cardiovascular disease. All embodiments of this group possesses the following common features: a active pharmaceutical ingredient of the drug is taking lncRNA DCRT with a sequence shown as SEQ ID NO.1 as target.

In specific embodiments, the drug for treating cardiovascular disease, comprising: a active pharmaceutical ingredient, characterized in that, the active pharmaceutical ingredient comprises: a substance regulating expression of lncRNA DCRT.

In some examples, said regulating refers to up-regulating; preferably, the substance regulating expression of lncRNA DCRT is selected from a group consisting of primers upregulating or overexpressing lncRNA DCRT, a recombinant expression vector upregulating or overexpressing lncRNA DCRT, and a transformant upregulating or overexpressing lncRNA DCRT;
preferably, primers upregulating or overexpressing lncRNA DCRT include:

forward primer:
(SEQ ID NO: 3)
5′-AGATCCTCTGCTGATGTCACTTG-3′,
reverse primer:
(SEQ ID NO: 4)
5′-AAAATGTTCCGCCCCTCC-3′;

preferably, the recombinant expression vector upregulating or overexpressing lncRNA DCRT refers to an expression vector that connects amplification products of primers upregulating or overexpressing lncRNA DCRT;

In specific embodiments, the expression vector is selected from: pAAV vector;
preferably, the transformant upregulating or overexpressing lncRNA DCRT is host transformed with the recombinant expression vector;
preferably, the host is selected from recombinant adenovirus or cell;
preferably, the drug further comprises: medicinal excipient;
preferably, the cardiovascular disease is dilated cardiomyopathy.

A more specific embodiment of this group provides a drug for treating dilated cardiomyopathy, in which all embodiments have the following features: the active pharmaceutical ingredient of the drug for treating dilated cardiomyopathy is taking lncRNA DCRT as the drug target, and its therapeutic effect for treating dilated cardiomyopathy is achieved by upregulating expression of lncRNA DCRT. The present invention found through experiments that sequence of lncRNA DCRT is highly conserved, and content of lncRNA DCRT in patients with dilated cardiomyopathy is significantly lower than that in control group. The lncRNA DCRT in main myocardial cells is significantly reduced. Subsequently, it's confirmed by animal experiments that upregulating lncRNA DCRT has an effect of improving central function impairment and myocardial cell contractility reduction induced by thoracic aortic constriction surgery, to achieve a goal of assisting in treating dilated cardiomyopathy.

In further embodiments, the active pharmaceutical ingredient of the drug include substances that upregulate expression of lncRNA DCRT; alternatively, the active pharmaceutical ingredient of the drug can degrade or inhibit a substance that downregulates expression of lncRNA DCRT. As a segment of RNA obtained externally in animals, lncRNA DCRT can be upregulated through molecular biotechnology methods to achieve therapeutic effects. Other methods, such as certain chemical molecules, can also be used to inhibit its degradation and achieve the same goal of upregulating its level.

In a preferred embodiment, the active pharmaceutical ingredient of the drug comprises a full-length fragment of sequence of lncRNA DCRT, thereby increasing its expression level and further treating it.

In other embodiments, the drug further comprises pharmaceutically acceptable excipients and/or reagents for buffering, synthesizing, and/or purifying the lncRNA DCRT sequence fragments. A person skilled in the art can add various pharmaceutically acceptable adjuvants/excipients to the anti dilated cardiomyopathy drug of the present invention according to objective needs, and make various dosage forms for easy sales or promotion.

In specific embodiments, the pharmaceutically acceptable excipients or pharmaceutical excipients are selected from: solvents, projectiles, solubilizers, cosolvents, emulsifiers, colorants, adhesives, disintegrants, fillers, lubricants, wetting agents, osmotic pressure regulators, stabilizers, flow aids, flavor correction agents, preservatives, suspension aids, coating materials, aromatics, anti adhesives, integrators, penetration enhancers, pH regulators, buffering agents Plasticizers, surfactants, foaming agents, defoamers, thickeners, encapsulating agents, moisturizers, absorbers, diluents, flocculants, anti flocculants, filter aids, release blockers.

A person skilled in the art can make routine selections or adjustments to pharmaceutical excipients based on actual production needs, combined with conventional technical means or basic knowledge of pharmaceutical production processes (such as the «Encyclopedia of Pharmaceutical Formulation Technology», «Pharmaceutical Formulation Technology», etc.), in order to produce drugs with different dosage forms, storage conditions, and shelf life. This is not a technical barrier for the person skilled in the art, and can be easily achieved.

Those skilled in the art can select and allocate above excipients, and make the lncRNA DCRT of this invention and/or the substances regulating expression of lncRNA DCRT into different dosage forms, such as powder, tablet, oral liquid, capsule, granule, spray, gel, paste, decoction, pill, spray, inhaler, aerosol, injection, etc.

### Examples Group 3. Method of Preparing Drug of the Present Invention

This group of embodiments provides a method of preparing a drug for treating cardiovascular diseases, characterized in that, comprising the following step: a substance upregulating or overexpressing lncRNA DCRT is used as the active pharmaceutical ingredient of the drug; sequence of lncRNA DCRT is shown as SEQ ID NO.1.

In specific embodiments, the method of preparing a drug for treating cardiovascular diseases also comprising the following step: a substance upregulating or overexpressing lncRNA DCRT is mixed with a medicinal excipient to produce the drug;
preferably, the substance upregulating or overexpressing lncRNA DCRT is selected from a group consisting of primers upregulating or overexpressing lncRNA DCRT, a recombinant expression vector upregulating or overexpressing lncRNA DCRT, and a transformant upregulating or overexpressing lncRNA DCRT; In preferred embodiments, primers upregulating or overexpressing lncRNA DCRT include:

forward primer:
(SEQ ID NO: 3)
5′-AGATCCTCTGCTGATGTCACTTG-3′,
reverse primer:
(SEQ ID NO: 4)
5′-AAAATGTTCCGCCCCTCC-3′;

preferably, a recombinant expression vector upregulating or overexpressing lncRNA DCRT refers to an expression vector that connects amplification products of primers upregulating or overexpressing lncRNA DCRT;

In specific embodiments, the expression vector is selected from: pAAV vector;
and/or, the transformant upregulating or overexpressing lncRNA DCRT is host transformed with the recombinant expression vector;
in specific examples, the host is selected from recombinant adenovirus or cell;
preferably, the cardiovascular disease is dilated cardiomyopathy.

The present invention provides use of a substance capable of upregulating the expression of lncRNA DCRT in preparing anti-dilated cardiomyopathy drug. Any act of packaging above substance in commercial packaging labeled with anti-dilated cardiomyopathy use for any scale falls within protection scope of the present invention.

The present invention provides a method for preparing an anti dilated cardiomyopathy drug, comprising: using a substance that can upregulate expression of lncRNA DCRT as the active pharmaceutical ingredient of the anti dilated cardiomyopathy drug.

The specific experimental operation steps of this group of examples are shown in experiment example 2 below.

Examples group 4. Screening Method for Drug of the present invention.

This group of examples provides A screening method of a drug for treating cardiovascular diseases. All examples of this group possesses the following common features, said method comprising the following step: screening candidate substance with lncRNA DCRT as target; sequence of lncRNA DCRT is shown as SEQ ID NO.1.

In further embodiments, a screening method for a drug for treating cardiovascular disease further comprises the following step: further screening substance regulating lncRNA DCRT expression from the candidate substance as active pharmaceutical ingredient of the drug;
preferably, said regulating refers to up-regulating;
preferably, the substance regulating lncRNA DCRT expression is selected from: substance upregulating or overexpressing lncRNA DCRT;
preferably, substance upregulating or overexpressing lncRNA DCRT is selected from: a group consisting of primers upregulating or overexpressing lncRNA DCRT, a recombinant expression vector upregulating or overexpressing lncRNA DCRT, and a transformant upregulating or overexpressing lncRNA DCRT;

In preferred embodiments, primers upregulating or overexpressing lncRNA DCRT include:

forward primer:
(SEQ ID NO: 3)
5′-AGATCCTCTGCTGATGTCACTTG-3′,
reverse primer:
(SEQ ID NO: 4)
5′-AAAATGTTCCGCCCCTCC-3′;

preferably, a recombinant expression vector upregulating or overexpressing lncRNA DCRT refers to an expression vector that connects amplification products of primers upregulating or overexpressing lncRNA DCRT;

In specific embodiments, the expression vector is selected from: pAAV vector;
preferably, the transformant upregulating or overexpressing lncRNA DCRT is host transformed with the recombinant expression vector;
in specific examples, the host is selected from recombinant adenovirus or cell;
preferably, the cardiovascular disease is dilated cardiomyopathy.

The most specific example of this group provides a screening method for anti dilated cardiomyopathy drug, comprising the following steps: detecting whether candidate substance can upregulate expression of lncRNA DCRT, and screening for substances that can promote expression of lncRNA DCRT.

The specific experimental operation steps of this example can be found in experiment example 2.

### Experimental Example 1. lncRNA DCRT Detection of Myocardial Tissue in Patients with Dilated Cardiomyopathy

Collecting heart tissues from 14 patients with dilated cardiomyopathy and 7 healthy heart transplant donors, and storing them in a −80° C. freezer. Adding 1 ml TRIZOL (Invitrogen company) to every 50 mg of heart, grinding, and extracting RNA. Using Nanodrop® ND-1000 to detect RNA quality.

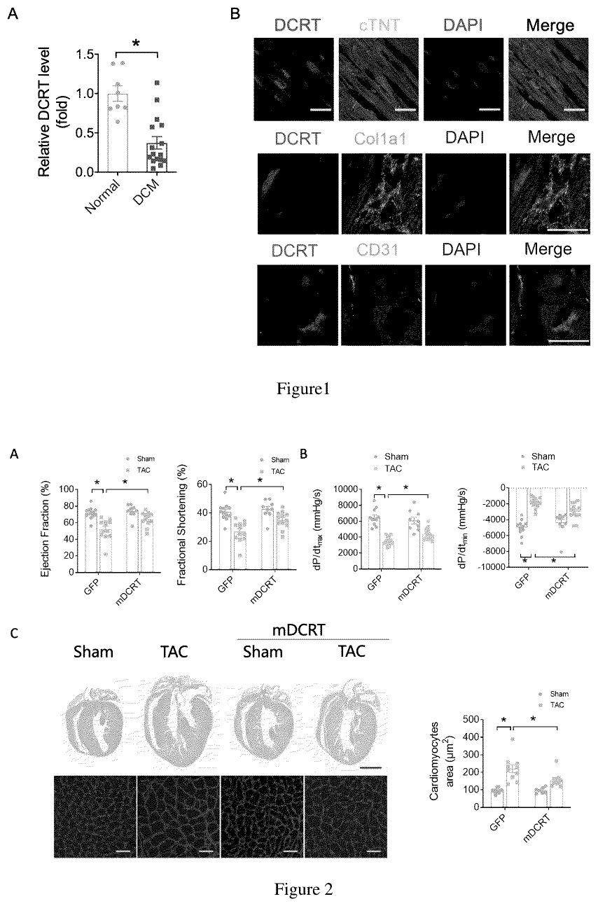

It's shown by results that the expression of lncRNA DCRT in the heart tissue of patients with dilated cardiomyopathy decreased (FIG. 1A); In cardiac tissue, lncRNA DCRT is mainly located in the nucleus of myocardial cells (FIG. 1B).

### Experiment Example 2. Detection of the Therapeutic Effect of lncRNA DCRT on Dilated Cardiomyopathy

1. Packaging and purification of overexpressing lncRNA DCRT transformants

This experimental example requires packaging two viruses: rAAV-GFP (negative control) carrying green fluorescent protein (GFP) and rAAV-DCRT (upregulated DCRT) carrying lncRNA DCRT; a reverse complementary sequence of lncRNA DCRT is shown as SEQ ID NO.2, which was delivered to Shanghai Sangon Biotechnology Service Co., Ltd to synthesize by adding Hind III and Not I adhesive ends to oligonucleotide single strand respectively. Annealing forms double chains, with the following system:
5×Annealing buffer 10 ul
Oligonucleotide positive chain (100 uM) 2.5 ul
Oligonucleotide negative chain (100 uM) 2.5 ul
Ultra pure water 35 ul

After mixing and centrifuging the system, carrying out annealing reaction on PCR instrument with reaction procedure of: 95° C. for 2 minutes, decrease by 1° C. every 90 seconds, decrease to 25° C., and reserve at 4° C.

### A. PAAV Vector Enzyme Digestion

Hind III (15 U/ul) 1 ul
Not I (10 U/ul) 1 ul
ten×K Buffer 2.5 ul
0.1% BSA 2.5 ul
DNA 5 ug
Filling up to 50 ul with ultrapure water
Mixing and centrifuging the system, and reacting at 37° C. for 2 hours.

### B. Gel Recycling and Purification

Putting a digestion product to 1% agarose gel for electrophoresis, cutting the gel block containing target fragment under ultraviolet light, and using a gel recovery kit to purify nucleic acid fragment with the following steps:
a) Weighing the gel block, adding a proper volume of GSB solution according to weight of the gel block, taking a water bath at 55° C. for 10 minutes, and mixing liquid upside down for three times until the gel is completely dissolved;
b) After cooling the liquid to room temperature, adding it to a centrifuge column and letting it stand for 1 minute. Centrifuging at 10000 g for 1 minute and discarding the liquid in collection tube;
c) 650 ul WB solution, centrifuging at 10000 g for 1 minute, discarding the liquid in collection tube;
d) Centrifuging at 10000 g for 2 minutes, placing the centrifuge column into a new EP tube, opening the lid, and letting it stand for 10 minutes;
e) Adding 50 ul of EB preheated at 65° C. to center of centrifuge column, letting it stand for 1 minute, centrifuging at 10000 g for 1 minute, and its eluent is the purified nucleic acid solution.

### C. Connection Between Target Fragment and Vector

vector 0.5 ul
Target fragment 8 ul
T4 ligase 0.5 ul
10×T4 ligase buffer 1 ul

Mixing and centrifuging the system (10 ul), and connecting it overnight at 16° C.

### D. Competent Cell Transformation

Taking T1 competent bacteria out of a −80° C. refrigerator and placing it on ice for 30 minutes to thaw. Adding above connecting products to the competent bacteria and placing on ice for 30 minutes. Heating them in a 42° C. water bath for 45 seconds, followed by an ice bath for 5 minutes. Adding 200 ul of antibiotic free LB medium (10 g/L tryptone, 5 g/L yeast extract, 10 g/L NaCl, pH 7.4) and shaking on a shaking table at 37° C. for 1 hour at 100 rpm. Spreading bacterial solution onto a solid LB (pancreatic peptone 10 g/L, yeast extract 5 g/L, NaCl 10 g/L, agar powder 15 g/L) culture plate containing 100 ug/ml ampicillin, and incubating overnight in a 37° C. incubator. Selecting the grown monoclonal colonies and amplifying them in liquid LB medium containing ampicillin. Sending the bacterial solution to Wuhan Tianyi Huiyuan Biotechnology Co., Ltd. for sequencing, and amplifying and preserving the correctly sequenced strains.

### Plasmid Extraction

Amplifying constructed pAAV-DCRT bacterial solution, pAAV-GFP bacterial solution, pXX9 bacterial solution, and phelper bacterial solution in TB medium containing 100 ug/ml ampicillin (tryptone 12 g/L, yeast extract 24 g/L, K₂HPO₄·3H₂O 16.43 g/L, KH₂PO₄ 2.31 g/L, 0.4% glycerol) for 14-16 hours. Extracting plasmids by using an endotoxin free plasmid extraction kit from Tiangen Biochemical Technology Co., Ltd with the following specific steps:
a) Centrifuging bacterial solution at 8000 g room temperature for 3 minutes to collect bacteria, removing supernatant, adding new bacterial solution, collecting bacterial body at bottom of centrifuge tube through repeated centrifugation, and removing supernatant thoroughly;
b) Adding an appropriate amount of P1 solution (first adding RNase A), and using a pipette and oscillator to completely resuspend bacterial precipitate;
c) Adding an appropriate amount of P2 solution, immediately inverting it 7 times, and letting it stand at room temperature for 5 minutes;
d) Adding an appropriate amount of P4 solution, immediately inverting it several times until flocculent precipitates appear in solution. Placing it at room temperature for 10 minutes, centrifuging at 8228 g at room temperature for 15 minutes, and filtering supernatant into a clean centrifuge tube by using filter CS1;
e) Adding isopropanol in a volume ratio of 1:0.3, mixing well, and transferring 10 ml to adsorption column CP6 (treated with equilibrium solution BL first). Centrifuging at room temperature of 8228 g for 2 minutes, discarding waste liquid in collection tube, and chromatographing the remaining solution through the column again;
f) Adding 10 ml of PW rinsing solution (adding ethanol first), centrifuging at room temperature of 8228 g for 2 minutes, and discarding waste liquid in collection tube;
g) Repeating step f;
h) Adding 3 ml of anhydrous ethanol, centrifuging at room temperature of 8228 g for 2 minutes, and discarding waste liquid in collection tube;
i) Centrifuging at room temperature of 8228 g for 5 minutes, placing adsorption column in a clean collection tube, opening lid and placing it 15 minutes;
j) Adding 1 ml of elution buffer TB to adsorption column, letting it stand at room temperature for 5 minutes, centrifuging at 8228 g room temperature for 2 minutes, and eluting adsorption column again with 0.5 ml TB. Transferring elution solution to an EP tube, and using NanoDrop2000 to detect concentration and purity of plasmid.

### E. Virus Packaging

This experiment used a three plasmid calcium phosphorus co transfection method to package virus in HEK 293T cells. Passaging HEK 293T in a cell culture dish with a diameter of 15 cm. After cells grew to about 85%, replacing complete culture medium. Adding each reagent to prepare plasmid transfection solution was prepared in the following order (for each culture dish):
CaCl₂) solution (0.25M) 2 ml
Phelper plasmid 30 ug
PXX9 plasmid 10 ug
PAAV-DCRT or pAAV-GFP plasmid 20 ug
HEBS solution*(slowly dropped) 2 ml
*HEBS solution (NaCl 280 mM, HEPES 50 mM, Na₂HPO₄ 1.5 mM, KCl 10 mM, D-glucose 12 mM, pH 6.80-7.05)

Droping above plasmid transfection solution into HEK 293T cell dishes, culturing for 12 hours, then replacing with high sugar DMEM medium containing 5% fetal bovine serum, continuing to culture for 48-60 hours, collecting cells, and storing them in a −80° C. freezer.

### F. Virus Purification

Repeatedly freezing and thawing cells between −80° C. and 37° C., centrifuging at 8000 g for 15 minutes, and transferring supernatant to a clean round bottom centrifuge tube. The remaining steps are as follows:
a) Adding 10% liquid volume of chloroform and placing it horizontally on a shaking table at 37° C., shaking at 200 rpm for 1 hour;
b) Adding NaCl to a final concentration of 1 mol/L, dissolving and mixing well, centrifuging at 4° C. for 15 minutes at 12000 g;
c) Transferring supernatant to a clean round bottom centrifuge tube, adding 10 g of PEG8000 to every 100 ml of liquid, shaking vigorously to dissolve and mix well, and taking an ice bath for 1 hour;
d) Centrifuging 11000 g at 4° C. for 15 minutes, discarding supernatant, and using pre cooled PBS (2×) to blow solution to dissolve precipitate, then dividing liquid into 1.5 ml EP tubes;
e) Adding 1 ug/ml of DNaseI and RnaseA and letting it stand at room temperature for 1 hour;
f) Adding an equal volume of chloroform, shaking and mixing well, then centrifuging at 4° C. for 5 minutes at 12000 g, and transferring supernatant to a clean EP tube to obtain the purified virus solution.

### G. Measuring Virus Titer

a) Taking 40 ul of virus solution into a clean EP tube, adding 5 ul of protease K (20 mg/ml), mixing well, and placing in a 55° C. water bath for 1 hour;
b) Adding 150 ul of ultrapure water and 200 ul of phenol:chloroform:isoamyl alcohol (25:24:1), shaking and mixing well, then centrifuging at 4° C. for 10 minutes at 12000 g;
c) Taking supernatant into a clean EP tube, adding an equal volume of chloroform, shaking well, and centrifuging at 4° C. for 5 minutes at 12000 g. Supernatant is viral nucleic acid;
d) Real-time quantitative PCR (CMV forward and reverse primers) method can detect viral nucleic acid fragment content to calculate viral titer. Primer sequences are shown as SEQ ID NO.3 and SEQ ID NO.4.
   3. Using SYBR Green Purchased from Thermofish Company to Perform Real-Time PCR Detection on Expression of lncRNA DCRT:

### Reverse Transcription:

### RT Primer Mix Composition:

Oligo RT Primer 1 μL
Random RT Primer 1 μL
RNase free H₂O 78 μL

### Reverse Transcription Reaction System:

RNA template 2 ug
RT Primer Mix 4 μL
RNase free H₂O up to 19 μL

After mixing above systems, centrifuging them instantaneously, incubating at 70° C. for 10 minutes, then icing them for 2 minutes, and then adding the following reagents:
2× TS reaction buffer 25 μL
TS enzyme 2.5 μL
RNase free H₂O 3.5 μL

### Reverse Transcription Reaction Program:

42° ° C. for 60 minutes, 70° ° C. for 10 minutes; After stopping, reserving at 4° C. and storing product at −20° C.

### Real Time PCR:

Reaction system: 2×SYBR Green Mix 9 μL
RT product 2 μL
LncRNA DCRT Forward Primer 2 μL
LncRNA DCRT Reverse Primer 2 μL
RNase free H₂O 5 μL

### Reaction Procedure:

95° C. 30 sec−(95° C. 10 sec−60° C. 20 sec−70° C. 1 sec)×40 cycles−Melting Curve

### Primer Sequence:

DCRT Forward Primer:
(SEQ ID NO: 3)
5′-AGATCCTCTGCTGATGTCACTTG-3′
DCRT Reverse Primer:
(SEQ ID NO: 4)
5′-AAAATGTTCCGCCCCTCC-3′.

2. Protective effect of lncRNA DCRT on cardiac function in mice with thoracic aortic constriction surgery induced cardiac dilation:

After high expression of lncRNA DCRT in mouse hearts, detecting cardiac function by using ultrasound and cardiac catheterization. It's shown by results that full length of lncRNA DCRT had an effect on improving central function impairment in a cardiac dilation model induced by thoracic aortic constriction surgery (FIGS. 2A and B). It's shown by FIG. 2A that, using mouse ultrasound detection, it was found that full length of lncRNA DCRT has effect of improving decrease in ejection fraction in a cardiac dilation model induced by thoracic aortic constriction surgery; it's shown by FIG. 2B that, it's found through cardiac catheterization detection that, full length of lncRNA DCRT has effect of improving central function impairment in a cardiac dilation model induced by thoracic aortic constriction surgery.

3. Protective effect of lncRNA DCRT on pathological increase of myocardial cells induced by thoracic aortic constriction surgery in mice with cardiac dilation:

After high expression of lncRNA DCRT in mouse hearts, measuring area of myocardial cells in mice. It's shown by results that, full length of lncRNA DCRT had an effect on improving the pathological increase in myocardial cell area induced by thoracic aortic constriction surgery. (FIG. 2C).

1. Use of lncRNA DCRT with sequence shown as SEQ ID NO.1 as drug.

2. The use of lncRNA DCRT with sequence shown as SEQ ID NO.1 as drug according to claim 1, characterized in that, a reverse complementary sequence of lncRNA DCRT is shown as SEQ ID NO.2;
and/or, the drug refers to a drug for treating cardiovascular disease.

3. The use of lncRNA DCRT with sequence shown as SEQ ID NO.1 as drug according to claim 2, characterized in that, the cardiovascular disease is dilated cardiomyopathy.

4. A drug for treating cardiovascular disease, characterized in that, its active pharmaceutical ingredient is taking lncRNA DCRT with a sequence shown as SEQ ID NO.1 as target.

5. The drug for treating cardiovascular disease according to claim 4, comprising: a active pharmaceutical ingredient, characterized in that, the active pharmaceutical ingredient comprises: a substance regulating expression of lncRNA DCRT.

6. The drug for treating cardiovascular disease according to claim 5, characterized in that, said regulating refers to up-regulating;
and/or, the substance regulating expression of lncRNA DCRT is selected from a group consisting of primers upregulating or overexpressing lncRNA DCRT, a recombinant expression vector upregulating or overexpressing lncRNA DCRT, and a transformant upregulating or overexpressing lncRNA DCRT;
and/or, primers upregulating or overexpressing lncRNA DCRT include:
forward primer:
(SEQ ID NO: 3)
5′-AGATCCTCTGCTGATGTCACTTG-3′,
reverse primer:
(SEQ ID NO: 4)
5′-AAAATGTTCCGCCCCTCC-3′;
and/or, the recombinant expression vector upregulating or overexpressing lncRNA DCRT refers to an expression vector that connects amplification products of primers upregulating or overexpressing lncRNA DCRT;
and/or, the expression vector is selected from: pAAV vector;
and/or, the transformant upregulating or overexpressing lncRNA DCRT is host transformed with the recombinant expression vector;
and/or, the host is selected from recombinant adenovirus or cell;
and/or, the drug further comprises: medicinal excipient;
and/or, the cardiovascular disease is dilated cardiomyopathy.

7. A method of preparing a drug for treating cardiovascular disease, characterized in that, comprising the following step: a substance upregulating or overexpressing lncRNA DCRT is used as the active pharmaceutical ingredient of the drug;
sequence of lncRNA DCRT is shown as SEQ ID NO.1.

8. The method of preparing a drug for treating cardiovascular disease according to claim 7, characterized in that, also comprising the following step: a substance upregulating or overexpressing lncRNA DCRT is mixed with a medicinal excipient to produce the drug;
and/or, the substance upregulating or overexpressing lncRNA DCRT is selected from a group consisting of primers upregulating or overexpressing lncRNA DCRT, a recombinant expression vector upregulating or overexpressing lncRNA DCRT, and a transformant upregulating or overexpressing lncRNA DCRT;
and/or, primers upregulating or overexpressing lncRNA DCRT include:
forward primer:
(SEQ ID NO: 3)
5′-AGATCCTCTGCTGATGTCACTTG-3′,
reverse primer:
(SEQ ID NO: 4)
5′-AAAATGTTCCGCCCCTCC-3′;
and/or, a recombinant expression vector upregulating or overexpressing lncRNA DCRT refers to an expression vector that connects amplification products of primers upregulating or overexpressing lncRNA DCRT;
and/or, the expression vector is selected from: pAAV vector;
and/or, the transformant upregulating or overexpressing lncRNA DCRT is host transformed with the recombinant expression vector;
and/or, the host is selected from recombinant adenovirus or cell;
and/or, the cardiovascular disease is dilated cardiomyopathy.

9. A screening method of a drug for treating cardiovascular disease, characterized in that, comprising the following step: screening candidate substance with lncRNA DCRT as target; sequence of lncRNA DCRT is shown as SEQ ID NO.1.

10. The screening method of a drug for treating cardiovascular disease according to claim 9, characterized in that, further comprising the following step: further screening substance regulating lncRNA DCRT expression from the candidate substance as active pharmaceutical ingredient of the drug;
and/or, said regulating refers to up-regulating;
and/or, the substance regulating lncRNA DCRT expression is selected from:
substance upregulating or overexpressing lncRNA DCRT;
and/or, substance upregulating or overexpressing lncRNA DCRT is selected from: a group consisting of primers upregulating or overexpressing lncRNA DCRT, a recombinant expression vector upregulating or overexpressing lncRNA DCRT, and a transformant upregulating or overexpressing lncRNA DCRT;
and/or, primers upregulating or overexpressing lncRNA DCRT include:
forward primer:
(SEQ ID NO: 3)
5′-AGATCCTCTGCTGATGTCACTTG-3′,
reverse primer:
(SEQ ID NO: 4)
5′-AAAATGTTCCGCCCCTCC-3′;
and/or, a recombinant expression vector upregulating or overexpressing lncRNA DCRT refers to an expression vector that connects amplification products of primers upregulating or overexpressing lncRNA DCRT;
and/or, the expression vector is selected from: pAAV vector;
and/or, the transformant upregulating or overexpressing lncRNA DCRT is host transformed with the recombinant expression vector;
and/or, the host is selected from recombinant adenovirus or cell;
and/or, the cardiovascular disease is dilated cardiomyopathy.
